(19) United States
(12) Patent Application Publication
(10) Pub. No.: US 2013/0245549 A1
(43) Pub. Date: Sep. 19, 2013

(54) BIORESORABLE DRUG DELIVERY MATRICES BASED ON CROSS-LINKED POLYSACCHARIDES, DOSAGE FORMS DESIGNED FOR DELAYED/CONTROLLED RELEASE

(71) Applicants: Garfield P. ROYER, Upperville, VA (US); Tatiana Bizikova, Frederick, MD (US)

(72) Inventors: Garfield P. ROYER, Upperville, VA (US); Tatiana Bizikova, Frederick, MD (US)

(73) Assignee: ROYER BIOMEDICAL, INC., Frederick, MD (US)

(21) Appl. No.: 13/612,247

(22) Filed: Sep. 12, 2012

Related U.S. Application Data
(60) Provisional application No. 61/534,767, filed on Sep. 14, 2011.

(51) Int. Cl. A61L 31/14

(52) U.S. Cl. CPC A61L 31/148
USPC 604/93.01; 424/426; 514/49; 514/274; 427/2.21

(57) ABSTRACT

Bioactive agents are embedded in a cross-linked dextran and coated with a bioresorbable polymer. When implanted in a mammal, the coated cross-linked dextran composition produces controlled release of the embedded bioactive agent.

### CROSS-REFERENCE TO RELATED APPLICATION

This application is related to and claims domestic priority benefits from U.S. Provisional Application Ser. No. 61/534,767 filed on Sep. 14, 2011, the entire content of which is expressly incorporated hereinto by reference.

### FIELD

Bioresorbable polymer matrices and their production and use as delivery systems for bioactive agents are provided. In certain exemplary embodiments, controlled release of pharmaceuticals and other bioactive agents is achieved with the use of the disclosed matrices.

### BACKGROUND AND SUMMARY

Various ways for delivery of pharmaceuticals in veterinary and human medicine are known, such as oral, topical, ocular, vaginal, rectal, buccal/sublingual, transdermal and parenteral (including for example intravenous infusion, I.M., S.C., or intra-articular injections and implants [e.g., S.C., intra-tumoral, peri-operative placement in post-resection cavities, placement of drug formulation on or within a wound, and implantation at an infection site]). The most popular route of drug administration is oral. This can be problematic in that many useful drugs such as aminoglycoside antibiotics are not orally active.

The dosage forms according to certain embodiments of the invention described herein include implants. Although effective systemic levels of medication can be attained via implants (such as s.c. products) some of the embodiments of the dosage forms described herein are designed for localized delivery.

Although non-resorbable polymers can be used to formulate advanced drug delivery systems, devices based thereon must be recovered, often via surgery. An example is antibiotic-containing beads of polymethylmethacrylate.¹ Resorbable matrices do not require a follow-up procedure which is advantageous in terms of patient convenience/compliance and cost. The lifetime in the body of the devices described herein is 4-6 weeks. The resorption occurs via hydrolysis and enzymatic degradation. Dosage form production is schematically illustrated in FIG. 1. ¹ Faisant N, Siepmann J, Benoit J P. PLGA-based microparticles: elucidation of mechanisms and a new, simple mathematical model quantifying drug release. Eur J Pharm Sci. 15, 355-66 (2002).

Polymeric dextran matrices of the variety shown schematically by FIG. 1 are described more completely in U.S. Pat. No. 8,039,021 to G.P. Royer, the entire content of which is expressly incorporated hereinto by reference.

There are a number of attractive features of this polymer matrix including:
1. Safe—non irritating and non-toxic
2. Not susceptible to proteolytic attack
3. Resorbable
4. Can deliver a wide range of active ingredients including small molecules, proteins and nucleic acids
5. Controllable release profile—including a lag period/delayed release
6. Stable
7. Amenable to cGMP manufacturing requirements

These and other aspects and advantages of the embodiments disclosed herein will be better understood by reference to the following detailed descriptions thereof.

### BRIEF DESCRIPTION OF THE DRAWINGS

FIG. 1 schematically depicts the formation of dextran matrix via dihydrazide cross-linking at pH 4-6;

FIG. 2 schematically depicts the preparation of coated spherical beads with delayed release capability;

FIG. 3 are photographs showing R-Gel spherical beads containing crystal violet dye;

FIG. 4 schematically depicts the preparation of PLGA tubes containing dextran matrix containing a bioactive agent;

FIG. 5 schematically depicts a theoretical treatment used to describe the Class II dosage form;

FIG. 6 is a release profile graph with a lag period obtained via equation (8) below; and

FIG. 7 is a graph of multiple classes of dosage forms that can be made to produce the profiles depicted.

### DETAILED DESCRIPTION

### Polymer Gel Formation

Syringe A contains oxidized dextran solution and Syringe B contains the solid mixture comprised of cross-linking reagent, dextran (native), and buffer components. Oxidized dextran is produced starting with USP dextran (M_{w}, 70,000; AMRESCO, Inc.). The polymer is oxidized with sodium(meta)periodate. Purification is accomplished with diafiltration. The resulting solution of oxidized dextran contains 150 mg/ml of polymer which has dialdehyde groups on 10% of the residues. The oxidation reaction may be represented schematically as:

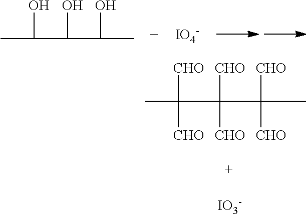

The cross-linking agent is adipic dihydrazide, shown below.

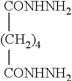

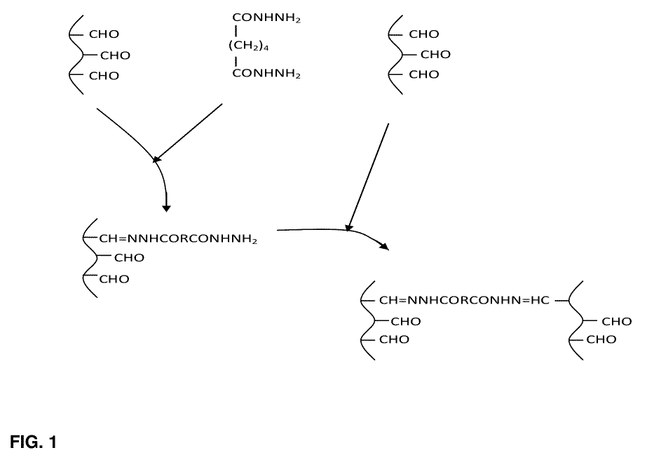

The gelation reaction occurs at a pH of 6 or below. This level of acidity precludes reaction of the aldehyde groups with amines which are charged at pH 6. The dihydrazides are very effective cross-linking reagents under these conditions in that they are not protonated and retain their nucleophilicity. The reaction involves initial addition of the hydrazide nitrogen to the aldehyde carbonyl carbon atom. The intermediate product subsequently dehydrates to form the hydrazone. Some internal cross-linking within a polymer molecule is inevitable but intermolecular cross-linking occurs sufficiently to form a strong gel. FIG. 1 illustrates the cross-linking reaction.

As mentioned elsewhere, the gelation reaction occurs as a result of cross-linking of oxidized dextran with the bi-functional hydrazide, adipic dihydrazide. This reagent was chosen because the reaction occurs at or below pH 6.0. At this pH the hydrazide (an alpha effect group) retains its nucleophilicity but the indigenous amino groups such as those on proteins are protonated and are thus unreactive with aldehydes or other electrophilic groups. This feature of the system has been proven with a study involving the release of azo-albumin from the dextran matrix prepared with 3% azoalbumin. All of the entrapped protein was recovered over a twelve day period. The gel remained in tact so the conclusion is that the protein was able to diffuse out of the matrix and was therefore not covalently bound to the matrix. Moreover, no local toxicity has been observed which is suggestive that the product is chemically inert. In other words, indigenous amino groups in host tissue do not react.

The gelation reaction is complete in 2 minutes. The gel is dimensionally stable and will not migrate. Theoretically, the polymer backbone is fixed so diffusion of oxidized dextran after the 2 minutes have elapsed is not possible. The process is isothermal and no host tissue/wound fluid or components are necessary for, or participate in, the reaction. The gelation reaction occurs on plastic, glass, underwater, or in air (drop suspended from the syringe tip). Various dosage form geometries have been produced with and without coatings.

### Formulation of Coated Products

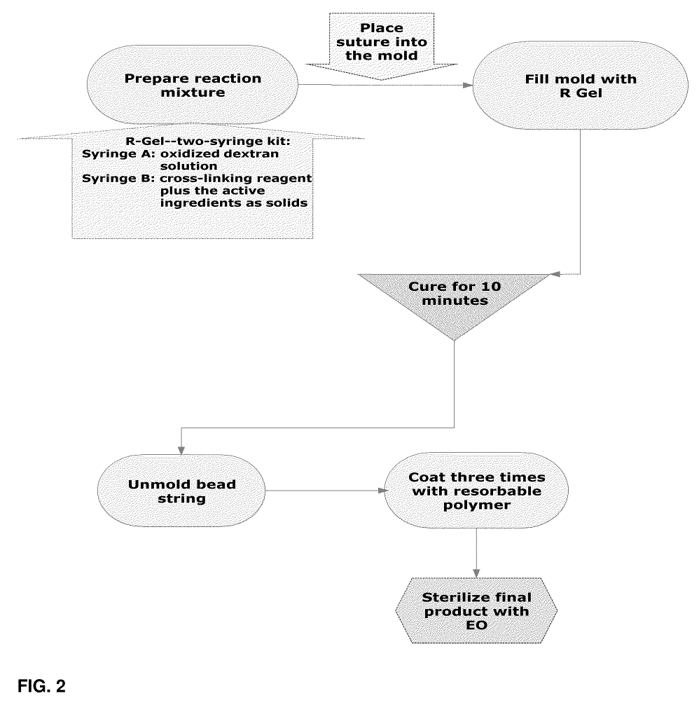

Approaches to the formation of coated dextran gel dosage forms are provided. According to some embodiments, spherical dextran beads in molds are provided (Class I). These beads may be connected by a resorbable suture. Following curing the string is dipped into a resorbable polymer solution for coating. FIG. 2 depicts the process schematically and this product is termed Class Ia. Generally, such process involves the following steps:
a. Prepare reaction mixture (sterile)
b. Fill mold containing suture
c. Cure for 10 minutes
d. Unmold bead string
e. Coat three times with resorbable polymer
f. Sterilize final product with EO

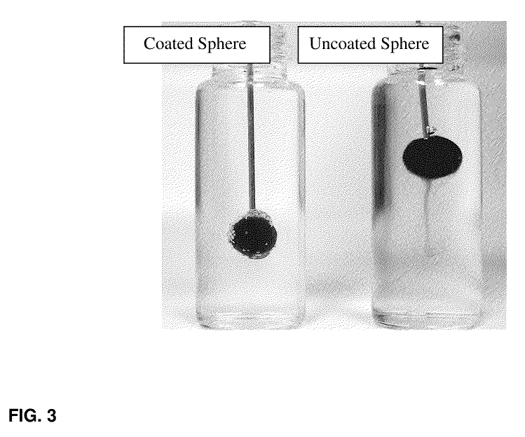

Another embodiment entails the use of a suture whereby the spherical beads are molded and then a syringe needle is inserted into the center of the bead (Class Ib). The coating is applied using the syringe needle as a handle. Removal of the needle produces a small hole through which the medicinal is released until the coating is degraded at which point there can be a delayed surge. The needle gauge will affect the initial release rate. The number and nature of coating layers will affect the timing of the surge phase. In this regard, FIG. 3 shows R-Gel spherical beads containing crystal violet dye. The polylactic acid coated bead did not release dye in the PBS buffer. The uncoated sphere started releasing violet dye immediately after it was completely submerged in the buffer.

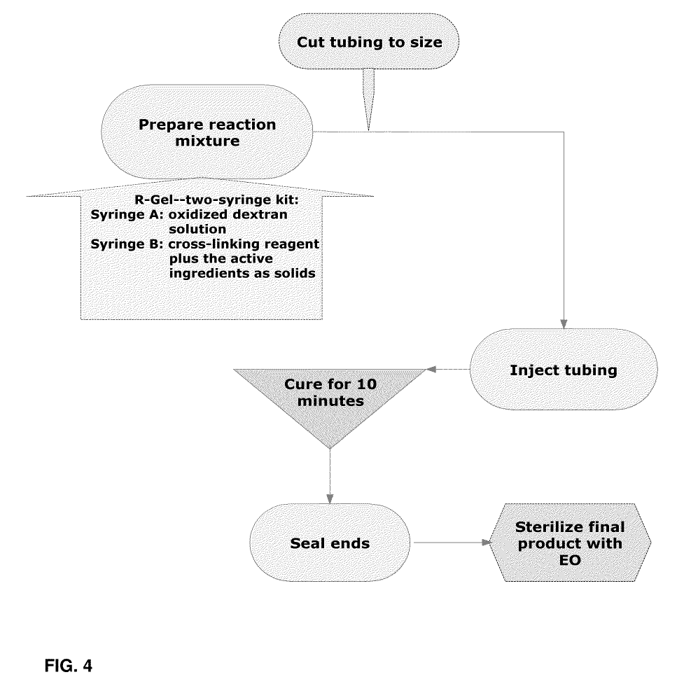

Another embodiment for achieving delayed-release dosage forms involves filling of PLGA tubes with the polymerizing mixture (Class II). Sterile tubes of PLGA are commercially available (Zeus MFR) in various diameters and wall thicknesses. The tubes are injected with dextran matrix containing a bioactive agent (FIG. 4). After curing (10 minutes) the ends are sealed. An alternative is to seal just one end or leave the ends open prior to implantation. A mixture of these dosage forms can also be employed to yield a delayed “burst” in release of drug following dissolution of the polymeric tubing. In general the process depicted by FIG. 4 comprises the following steps:

a. Prepare sterile reaction mixture
b. Cut tubing to size
c. Inject tubing
d. Cure
e. Seal ends
f. Sterilize using ethylene oxide

### Release Kinetics

Release of bioactive agents can be understood in view of the following analysis.

According to Fick's law the diffusion rate is given by

Rate=AD(∂[m]/∂x)

A represents the area which depends on the geometry of the dosage form and ∂[m]/∂x is the concentration gradient of the medicinal at the dosage form boundary.

D can be expressed as a variation of the Stokes-Einstein equation

D=kS/vM_{w}

in which k is a constant, S is the solubility of the medicinal, v is the viscosity, and M_{w} is the molecular weight of the medicinal. The relative low solubility of the active ingredient would contribute to prolonged release. The cross-linked polymer network potentially slows the release by affecting the viscosity of the medium. The concentration of polymer and the degree of cross-linking are variables which allow for viscosity control.

TABLE I
Control of the Release Profile

| Parameter | Variable |
|---|---|
| Area | Dosage form geometry |
| | Coating Class I |
| | Needle gauge used in Class 1b |
| | Wall thickness Class II |
| Solubility | Use of counter ions that affect solubility of the active agent |
| Viscosity | Polymer concentration and degree of cross-linking |
| Mw | The “effective” molecular weight of the medicinal can be |
| | increased by using a complexing agent |
| Coating | Nature and thickness of the coating determines lag time |

Release kinetics with coated dosage forms involves a lag period which appears when the effective surface area is increased and the surface erosion occurs. Polymers such as those listed in Table II are hydrolyzed in the body to produce metabolizable products.

TABLE II
Resorbable Polymers usable as coatings

Polylactic acid—PLA
Polylactic/glycolic acid—PLGA
Polyglycolic Acid—PGA
Polycaprolactone—PCL
Various polyanhydrides
Polyketals

Polylactic acid for example is resorbed as shown in the following reaction

PLA+H₂0→→lactic acid

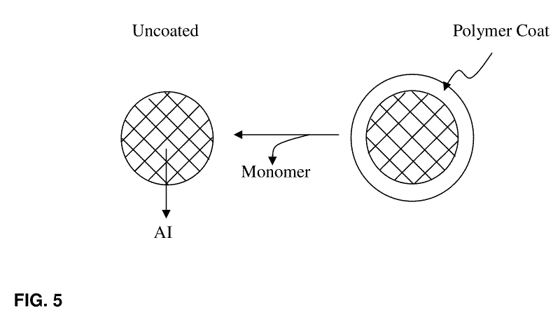

The rate of resorption of these polymers is dependent on the composition and molecular weight. The hydrolysis reaction is first order.² The theoretical treatment shown in FIG. 5 is used to describe the Class II dosage form but it is generally applicable. ² Banu S. Zolnik, Diane J. Burgess, Effect of acidic pH on PLGA microsphere degradation and release. J Control Release. 122, 338-44 (2007).

PDLGA has a residence time in the body of 1-2 months. When both ends of the tubes are closed the drug release starts when the polymer is sufficiently eroded. As shown above the rate of drug release will depend on open surface area, A, which is dependent on the rate of polymer degradation:

$${Polymer}_{P} {}^{k} {Monomer}_{M} ( or soluble oligomer ) \qquad ( 1 )$$

This process is dependent on the type of polymer, molecular weight, and the thickness of the PDLGA tube. This tubing is available from Zeus, Inc. of Orangeburg, S.C. in a variety of geometries and polymer compositions.

The fraction of accessible surface is dependent on the extent of polymer degradation.

A/A_{T}=[M]/Pₒ (2)

A_{T} is the total attainable surface area and Pₒ is the starting amount of polymer (both known).

For reaction (1)

$$\begin{matrix}rate = - d [ P ] / d t = d [ M ] / d t = k ( {P}_{o} - [ M ] ) & ( 3 ) \\ {\int }_{0}^{t} d [ M ] / {P}_{o} [ M ] = {\int }_{0}^{t} k d t \\ or \\ ( {P}_{o} - [ M ] ) / {P}_{o} = {e}^{- kt} & ( 4 )\end{matrix}$$

Combination of (2) and (4) yields (5) as [M]/Po=(1−e^{-kt})

A=A_{T}[1−e^{-kt}] in which A_{T} is the surface area of uncoated dosage form; k is the rate constant for polymer degradation (5)

Fick's first law can be stated as follows

dm/dt=DA(∂[m]/∂x)=D₁A; D1=D(∂[m]/∂x) (6)

In the early stages of release of the active ingredient, m, we assume that (∂[m]/∂x) is constant at the dosage form boundary.

Combination of (5) and (6) gives

dm/dt=D₁A_{T}(1−e^{-kt})=D₂(1−e^{-kt}); D2=D₁A_{T} (7)

Integration of (7) yields

m=D₂t+D₂/k[(e^{-kt}−1)] (8)

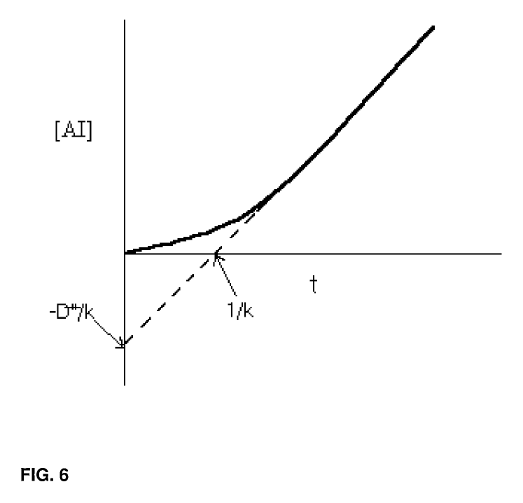

Equation (8) produces a release profile with a lag period as shown in FIG. 6. The of intercept 1/k shown in FIG. 6 is related to the half-time for polymer erosion

1/k=t_{1/2}/0.69 (9)

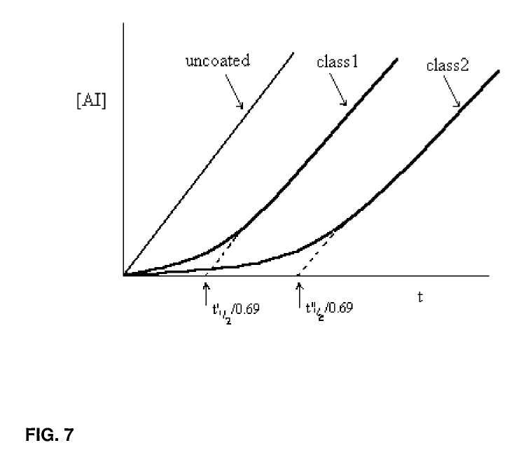

So the lag period is dependent on the half-time associated with degradation of the polymer layer which is an adjustable parameter. Hence multiple classes of dosage forms can be made to produce the profiles shown in FIG. 7.

Composition and thickness of the layer can be varied to produce a wide range of lag times. PDLGA is a good candidate for the polymer coating. Variation of coating thickness, molecular weight, and L/G ratio will produce different lag times as a consequence of slower degradation of the coating.

### EXAMPLES

### Delayed Release Drug Delivery

5-FU is of interest for treatment of glioblastoma using intracranial placement of R Gel 5-FU. It is useful in R Gel for intra-tumoral treatment of cancer.

### Example 1

### Release Profile—21 Day Release

Double syringe system is used in preparation of R Gel 5-FU Spheres. One syringe contains a polymer solution such as oxidized dextran. In the second syringe is a mixture of solid drug and solid dihydrazide. Two component buffer is included to control pH. A diluting agent is also added into the second syringe. The two syringes are coupled and the contents are mixed by reciprocation. Initially, the viscosity is low which permits the product to inject into the mold.

Various forms of R-Gel 5FU can be produced. One approach is to inject the gel into the mold with spherical or cylindrical cavities. The cavities within the mold are connected by a tunnel. The resorbable surgical suture is placed through the tunnels connecting the cavities in order to create a string of beads. R-Gel is allowed to set up in the mold. Solidification occurs within 2 minutes. The mold is then open and spheres are removed. The compact spheres are coated by dipping (immersion and withdrawal) into a polymer solution containing a biodegradable polymer (polylactic acid, polycaprolactone).

5 FU (140 mg) was placed into a porcelain mortar and mixed thoroughly along with 50 mg of Dextran 70, adipic acid dihydrazide (20 mg) and mixture of sodium succinate (3.5 mg) and succinic acid (1.5 mg). The material was then transferred into a 3 ml syringe (female Luer lock). Oxidized dextran solution (Mw 70,000; 150 mg/ml; 1 ml) was drawn into another syringe (male Luer lock). The syringes were connected and the contents were mixed by reciprocation (about 20 times). The homogenous suspension was injected into a mold with spherical holes (7 mm in diameter). After 10 minutes the mold was open and the spheres were removed. The R-Gel spheres were coated (3×) by dipping the spheres into the polymer containing solution (1 g polylactic acid per 2 ml of acetone). The coated spheres were allowed to air-dry overnight.

The R-Gel 5FU sphere was transferred into a 2 ml centrifuge tube for the release experiment in 1 ml PBS buffer.

### Release Profile

| Day % | Released |
|---|---|
| 1 | 0.6 |
| 2 | 0.3 |
| 3 | 0.9 |
| 4 | 4.3 |
| 5 | 2.6 |
| 6 | 1.7 |
| 7 | 2.3 |
| 8 | 2.9 |
| 9 | 3.1 |
| 10 | 4.3 |
| 11 | 5.3 |
| 12 | 4.4 |
| 13 | 5.7 |
| 14 | 6.9 |
| 15 | 8.9 |
| 16 | 10.9 |
| 17 | 11.0 |
| 18 | 12.4 |
| 19 | 6.0 |
| 20 | 0.9 |
| 21 | 0.6 |
| 22 | 0.0 |

### Example 2

The dry mixture of 5 FU (150 mg), adipic acid dihydrazide (20 mg), sodium succinate (3.5 mg) and succinic acid (1.5 mg) was placed into a 3 ml syringe (female Luer lock). The syringe with the dry mixture was connected to a second syringe (male Luer lock) containing oxidized dextran solution (Mw 70,000; 150 mg/ml; 1 ml). The contents of both syringes were mixed by reciprocation (about 20 times). Sterile PLGA tubes (internal diameter=1.6 mm) were cut to a length of 1.5 cm. The homogenous suspension (80 μl) was injected into each tube. After curing (10 minutes), the ends of one tube were sealed. The second tube was sealed just from one end. The ends of the third tube were left open.

The tubes with R-Gel 5FU were transferred into a 5 ml glass vial for the release experiment in 1 ml PBS buffer.

| | R-Gel 5FU | | |
|---|---|---|---|
| | Tube I | R-Gel 5FU | R-Gel 5FU |
| | (unsealed) | Tube II (one end sealed) | Tube III (sealed) |
| % Released/ | 22.5 | 5.8 | 0 |
| first day | | | |

### Example 3

Capecitabine (400 mg) was placed into a porcelain mortar and mixed thoroughly along with adipic acid dihydrazide (20 mg) and mixture of sodium succinate (3.5 mg) and succinic acid (1.5 mg). The material was then transferred into a 3 ml syringe (female Luer lock). Oxidized dextran solution (Mw 70,000; 150 mg/ml; 1 ml) was drawn into another syringe (male Luer lock). The syringes were connected and the contents were mixed by reciprocation (about 20 times). Sterile PLGA tubes (internal diameter=1.6 mm) were cut to a length of 1.5 cm. The homogenous suspension (80 μl) was injected into each tube. After curing (10 minutes), the ends of one tube were sealed. The second tube was sealed just from one end. The ends of the third tube were left open.

The tubes with R-Gel Capecitabine were transferred into a 5 ml glass vial for the release experiment in 1 ml PBS buffer.

| | R-Gel | R-Gel | R-Gel |
|---|---|---|---|
| | Capecitabine | Capecitabine | Capecitabine |
| | Tube I | Tube II | Tube III |
| | (unsealed) | (one end sealed) | (sealed) |
| % Released/first day | 10.3 | 4.3 | 0 |
| % Released/second day | 7.2 | 2.5 | 0 |
| % Released/third day | 4.1 | 1.6 | 0 |

1. A composition comprising a bioactive agent embedded in a cross-linked dextran and coated with a bioresorbable polymer, wherein when implanted in a mammal, said composition produces controlled release of the bioactive agent.

2. A composition as in claim 1 which is in the form of spherical beads coated with a bioresorbable polymer.

3. A composition as in claim 1 which is in the form of a tube made of a bioresorbable polymer.

4. A composition as in claim 1 comprising a mixture of spherical beads that have coatings with different degradation rates.

5. A composition as in claim 1, wherein the polymer is selected from the group consisting of PLA, PLGA, PGA, PCL, polyanhydrides and polyketals.

6. A tubular product comprising the composition of claim 1 wherein the coating is the shape of a tube.

7. A tubular product as in claim 6, wherein one or both ends of the tube are open.

8. A tubular product as in claim 6, wherein neither end of the tube is open.

9. A tubular product as in claim 6 which is amenable to cutting prior to implantation, wherein the number of cuts affects the bioactive agent release profile.

10. A method of treating cancer in a mammal comprising:
delivering a radiation sensitizer in a composition as in claim 1 to a post-resection cavity, and
administering radiation to the mammal,
wherein the release of radiation sensitizer in the mammal is synchronized with the treatment of the mammal with radiation.

11. A method as in claim 10 wherein the chemotherapeutic agent is selected from 5-FU, taxol, taxotere, doxorubicin, capecitabine or cisplatin.

12. A method of treating cancer in a mammal comprising:
delivering a chemotherapeutic agent in a composition as in claim 1 to a post-resection cavity.

13. A method as in claim 12 wherein the chemotherapeutic agent is selected from 5-FU, taxol, taxotere, doxorubicin, capecitabine or cisplatin.

14. A method of treating a localized infection comprising administering the composition of claim 1 to the infection wherein the bioactive agent is an antibiotic.

15. A method of treating acute or chronic osteomyelitis comprising administering the composition of claim 1 wherein the bioactive agent is an antibiotic to the infected area.

16. A method of treating an infected prosthetic joint in a mammal comprising administering the composition of claim 1 to the infected area wherein the bioactive agent is an antibiotic.

17. A method as in claim 16 wherein the prosthesis is removed prior to administering the composition.

18. A method as in claim 16 wherein the composition is administered by endosteal implantation.

19. A method as in claim 16 wherein the bioactive agent is a hormone.

20. A method of providing controlled release of a bioactive agent comprising administering the composition of claim 1 to a mammal wherein the composition is in various coated dosage forms to provide a substantially constant and controlled release of the bioactive agent.

21. A method as in claim 20 wherein the administering is implanting subcutaneously.

22. A method as in claim 21 wherein the controlled release is sustained 4-6 weeks.

23. A method as in claim 20, wherein the composition is tubular in shape.

24. A method as in claim 20 wherein the administration is by a needle or trocar.

25. A method as in claim 20 wherein an uncoated dosage form is combined with a coated dosage form whereby the resultant profile is substantially constant.

26. A method as in claim 20, wherein an uncoated dosage form is combined with a coated dosage form whereby the resultant profile is either biphasic or polyphasic.

27. A method of making a composition as in claim 1 comprising coating a bioactive agent embedded in a cross-linked dextran with a bioresorbable polymer.

28. A composition as in claim 1 wherein the bioactive agent embedded in a cross-linked dextran is the reaction product of a reaction mixture comprised of:
an oxidized dextran solution,
a cross linking hydrazide, and
a bioactive agent, wherein
the oxidized dextran has a molecular weight of 40,000 or greater, and wherein
the reaction product is a hydrazide cross-linked oxidized dextran matrix with the bioactive agent entrapped therein, and wherein
the matrix solidifies within about 1 to about 10 minutes.

29. The composition as in claim 28, wherein the cross-linking hydrazide comprises adipic dihydrazide.

30. The composition as in claim 28, wherein the cross-linking hydrazide is at least one dihydrazide selected from the group consisting of succinic acid dihydrazide, glutaric acid dihydrazide, adipic acid dihydrazide, pimelic acid dihydrazide, suberic acid dihydrazide, azelaic acid dihydrazide, sebacic acid dihydrazide, undecanedioic acid dihydrazide, dodecanedioic acid dihydrazide, bras sylic acid dihydrazide, tetradecanedioic acid dihydrazide, pentadecanedioic acid dihydrazide, thapsic acid dihydrazide, octadecanedioic acid dihydrazide.

31. The composition as in claim 1, further comprising a release agent for controlling release of the bioactive agent from the composition.

32. The composition as in claim 1, wherein the bioactive agent comprises of least one selected from osteoinductive agents, antibiotics, anesthetics, growth factors, cells, anti-tumor agents, anti-inflammatory agents, antiparasitics, antigens, adjuvants, cytokines and hormones.

33. The composition as in claim 1, wherein the bioactive agent is an antibiotic selected from the group consisting of amikacin, clindamycin, tobramycin, ciprofloxacin, piperacillin, ceftiofur, vancomycin, doxycycline, gentamicin, levofloxacin and fluoroquinolones.

34. A composition comprising a bioactive agent embedded in a cross-linked aldehydic polymer and coated with a bioresorbable polymer, wherein when implanted in a mammal, said composition produces controlled release of the bioactive agent.

35. A method of preventing infections of a surgical wound comprising administering the composition of claim 1 to the surgical wound, wherein the bioactive agent is an antibiotic.
